(19) United States
(12) Patent Application Publication
(10) Pub. No.: US 2023/0320813 A1
(43) Pub. Date: Oct. 12, 2023

(54) ENDOSCOPE CLEANING DEVICE

(71) Applicant: United States Endoscopy Group, Inc., Mentor, OH (US)

(72) Inventors: Christopher J. Kaye, Middleburg Heights, OH (US); Scott Haack, Chardon, OH (US)

(73) Assignee: United States Endoscopy Group, Inc., Mentor, OH (US)

(21) Appl. No.: 18/190,525

(22) Filed: Mar. 27, 2023

Related U.S. Application Data
(63) Continuation of application No. 16/508,030, filed on Jul. 10, 2019, now abandoned.
(60) Provisional application No. 62/696,270, filed on Jul. 10, 2018.

(51) Int. Cl. A46B 5/06; A46B 9/02; A61B 90/70

(52) U.S. Cl. CPC A61B 90/70; A46B 5/06; A46B 9/02; A46B 2200/3013; A46B 2200/3093; A61B 2090/701

(57) ABSTRACT

An apparatus and method for using an endoscope pre-cleaning and cleaning device. The device includes an elongated and flexible substrate and an abrasive element running along the length of the substrate. The abrasive element extends in an outward direction from the substrate and can have bristles in a continuous line along the substrate. The bristles can be various lengths, thicknesses, materials, and grouped in various densities along the substrate.

### CROSS-REFERENCE TO RELATED APPLICATIONS

The present application is a continuation of U.S. Pat. Application No. 16/508,030, filed Jul. 10, 2019, which claims benefits and priority to U.S. Provisional Pat. Application No. 62/696,270, filed on Jul. 10, 2018, the entire disclosures of which are incorporated herein by reference.

### FIELD OF THE INVENTION

The various embodiments relate to an elongated substrate that can reside in an endoscope channel to deliver an antimicrobial to the channel surface. The product geometry can incorporate one or more external abrasive elements attached to it to provide continuous abrasive cleaning action of a channel in an endoscope while it is pushed in and/or pulled through the channel. The elongated substrate and abrasive element can aggressively loosen debris from within the channel, also working in concert with residual moisture for the effective activation of the antimicrobials to the interior of the channel. The device described herein provides contact with the internal endoscope channel over its entire length to deliver antimicrobials.

### BACKGROUND INFORMATION

Endoscopic devices are well-known in the medical arts and are commonly used for numerous medical procedures. One exemplary procedure is removing polyps, lesions or other types of targeted tissue from the gastrointestinal wall of a human subject. During this and other endoscopic procedures, the exterior and interior portions of the endoscope are typically contaminated. Devices which aid in the effective cleaning of the endoscope can help to improve the quality of the preparation of the endoscope for future procedures as well as lengthen the life of the endoscope. There is a need for devices having an increased amount of contact with the interior surface of the channel for thorough cleaning of the endoscope channels.

Immediately after a medical procedure using an endoscope or similar device, the device is typically soiled and requires both cleaning and sterilization or high-level disinfection (HLD). The current practice regarding endoscopes is to perform a pre-cleaning procedure at the site of an endoscopic procedure, at the end of the endoscopic procedure, before removing the endoscope from the procedure room, as described in Standards of Infection Control in Reprocessing of Flexible Gastrointestinal Endoscopes, by the Society of Gastroenterology Nurses and Associates, Inc. A typical pre-cleaning procedure consists of suctioning a large volume of appropriate detergent solution through the endoscope until the endoscope is clear, and then is completed by suctioning air. Next, the channel is flushed with a water rinse and then forced air is pushed through the channel to drive water from the channel. The use of detergent is optional, and the guidelines do not recommend a specific detergent or type for this approach. At least one endoscope manufacturer recommends the pre-cleaning step be accomplished with water only (followed by forced air, but no detergent). While the detergent fluid can assist in releasing some of the biological fluid on the scope channel, it is inadequate to loosen debris which is more viscous and gelatinous or even solid. In addition, delays in performing the pre-cleaning step can result in contaminants and debris more aggressively adhering to the endoscope channel. Any residual water or moisture left in the channel could also cause potential biological growth of contaminants that were/are present in the channel.

Manual cleaning of endoscopes prior to automated/manual high-level disinfection or sterilization is an important step in removing the microbial burden from an endoscope. Retained debris can contribute to biofilm development (Fang et al., 2010, “A study of the efficacy of bacterial biofilm cleanout for gastrointestinal endoscopes”) and can interfere with the high-level disinfectant capability to effectively kill and/or inactivate microorganisms (Roberts, 2013, “The role of biofilms in reprocessing medical devices”).

Manual cleaning and thorough brushing of channels should be performed even when automatic endoscope reprocessor (AER) manufacturers claim that manual cleaning is unnecessary (Muscarella, “Inconsistencies in Endoscope-Reprocessing and Infection-Control Guidelines: The Importance of Endoscope Drying,” American Journal of Gastroenterology, 2009).

After each passage, the brush is typically rinsed in a detergent solution, to remove any visible debris before the brush is reinserted. The brushing step can be continued until there is no debris visible on the brush.

There is a need for cleaning devices to clean the channels of endoscopes having an increased amount of contact with an interior surface of the channel. Existing devices interact with the interior surface of an endoscope channel over a relatively short distance when compared to the entire length of the channel because they have a typical brush length of less than one inch. There is a need for cleaning brushes that make continuous contact with the interior of the endoscope channel as the brush migrates through the endoscope channel. The exemplary embodiments described herein provide continuous contact over the entire length of the endoscope channel and provide more aggressive interaction with debris in the channel.

### SUMMARY OF THE INVENTION

The present application describes a cleaning device for use to pre-clean and clean the interior portions of an endoscopic device.

In view of the foregoing, one or more embodiments of a device for pre-cleaning and/or cleaning the interior portions of an endoscopic device can include an elongate substrate with a length, and one or more abrasive elements that co-extend along the length of the elongate substrate. The abrasive element can be one or more of bristles, fibers, yarn, felt, or polymeric fin. The abrasive elements can be in a continuous line or can be grouped in bunches having gaps in between each bunch, in a line. The abrasive elements can extend in a single direction from the substrate or multiple directions from a twisted substrate. For example, the abrasive element can spiral around the outside of the substrate, such that its spiral wraps around the outer surface of the substrate, having the appearance that the abrasive element rotates around the substrate as it extends along the length of the substrate. The substrate can have a groove running along the length of the substrate. The abrasive elements can extend from all sides of a centered substrate, like a radius, or the abrasive elements can extend from just one or some sides of a substrate. The device can be flexible. For example, it needs to be flexible enough to move through the entire length of the endoscope without excessive drag, even when it is in a coiled position. The one or more abrasive elements can each extend outward in a single direction from the substrate when in a straightened configuration, and the device can have properties that induce it to twist and rotate along its length during its removal, so that the abrasive elements contact more surface area of the endoscope channel in a cork- screwing manner than might occur if provided in a straight configuration. The substrate can also have abrasive properties. The abrasive properties can result from an abrasive feature that can be a physical alteration to the substrate.

In an exemplary embodiment, a method of making a pre-cleaning and/or cleaning device for an endoscopic channel can include the steps of extruding a substrate and inline production of adding the abrasive elements. Both the substrate and the abrasive elements can contain antimicrobial additives. This device allows the method of pre-cleaning an endoscope channel by inserting the device into the channel, while the endoscope is still in the operating or procedure room, by pushing it or pulling it from one or both ends of the device. The method can include the step of leaving the device within the channel until the endoscope can be moved to another location for the more thorough manual cleaning, prior to AER. This step of inserting a cleaning device that contacts the internal channel walls of the endoscope has a distinct advantage over merely flushing fluids through the channel. An accurate comparison would be comparing the effectiveness of merely rinsing an item (for example dishes, windshield, etc.) to following this rinse with frictional contact.

While using a typical cleaning brush on the market today would provide some improvement in this regard, the exemplary embodiment has several features that render it superior. The exemplary embodiments described herein may not make full circumferential contact the entire time the device travels through the channel, but the properties that make it twist and snake through the channel provide a degree of contact continuously over its entire length and provide for more aggressive interaction with the debris in the channel. First, because the fibers and backside geometry run the entire length of the device, they also create contact with the channel walls over the entire length. Twisted wire brushes and other endoscope channel cleaning devices are typically between 0.50 to 1 inch in length, with the longest less than 6 inches. In addition, on the current devices, the catheter to which the brush or a pull-through elastomer feature is attached utilizes the catheter more as a conduit vehicle rather than a feature that assists in cleaning. The catheter is significantly smaller than the channel through which it travels. Since the catheter would not be in intimate contact with most of the channel, adding antimicrobials would have little to no beneficial effect. The preferred embodiment, in contrast, with its long fibers over the length of the device, will be in essentially intimate contact with the endoscope channel over the entire channel length. In addition, any residual moisture droplets left in the endoscope channel will interact with the substrate and fibers and be smeared and diffused, helping to initiate the antimicrobial additives that are incorporated into the device.

In another exemplary embodiment, the endoscope cleaning device can be connected to a device that provides vibrational and/or translational energy to the substrate. The vibrational and/or translational energy can be transmitted to the abrasive element(s).

It should be noted that an alternative embodiment would be to include an element having absorptive and/or compressive properties, like a sponge, as an abrasive element that extends over the entire length of the device. The compression properties help the abrasive element maintain intimate contact with the endoscope channel wall to deliver antimicrobials and possesses properties that would allow it to be inserted and removed without extraordinary effort.

The other advantage of leaving the device in the endoscope channel is a visual cue that the device is still dirty and is not to be used on another patient until cleaning has been completed. The method can include the steps of removing the device from the channel and rinsing the device (or possibly reapplying disinfectant to it), prior to inserting it again. Or the method can include moving the device in one or two directions. The method can include inserting the device in the channel and pushing or pulling it through so that the abrasive elements and/or abrasive feature removes debris from the inside of the channel. The push/pull technique can be enhanced by joining the ends together in a conveyor belt fashion. In this exemplary embodiment, the device’s total length would need to be a little more than twice the length of a typical endoscope device, to be linked and used effectively. In an exemplary embodiment of using the device, insertion of the device can be performed with another device either having counter-rotating wheels or one drive wheel and one that freely rotates that can automatically push and/or pull the device through the endoscope channel. This can be used with an exemplary embodiment having two ends of the endoscope cleaning device linked together.

These and aspects of the exemplary embodiments will become apparent from the following detailed description, taken in conjunction with the accompanying drawings, illustrating by way of example the principles of the various exemplary embodiments.

### BRIEF DESCRIPTION OF THE DRAWINGS

In order to facilitate a fuller understanding of the exemplary embodiments, reference is now made to the appended drawings. These drawings should not be construed as limiting and are intended to be exemplary only.

FIG. 1A is a perspective view of a cleaning device in a straightened configuration in accordance with an exemplary embodiment;

FIG. 1B illustrates a view of an exemplary embodiment of a cleaning device in a coiled configuration;

FIG. 2 is a schematic of a cleaning device in accordance with an exemplary embodiment;

FIG. 3A is a side view of the cleaning device of FIG. 1;

FIG. 3B is a detailed side view of a portion of a cleaning device in accordance with an exemplary embodiment;

FIG. 3C is a close-up view of an abrasive element of a cleaning device in accordance with an exemplary embodiment;

FIG. 4A is a cross-sectional view of a portion of a cleaning device in accordance with an exemplary embodiment;

FIG. 4B is a cross-sectional view of a portion of a substrate in accordance with an exemplary embodiment;

FIG. 5 is a perspective view of a portion of a cleaning device in accordance with an exemplary embodiment;

FIG. 6 is a side view of an end portion of a cleaning device in accordance with an exemplary embodiment;

FIG. 7A is a side view of an end portion of a cleaning device in accordance with another exemplary embodiment;

FIG. 7B is a cross-sectional view of a cleaning device in accordance with the exemplary embodiment illustrated in FIG. 7A;

FIG. 8A is a perspective view of an end portion of a cleaning device in accordance with another exemplary embodiment;

FIG. 8B is a cross-sectional view of a cleaning device in accordance with the exemplary embodiment illustrated in FIG. 8A;

FIG. 9A is a cross-sectional view of a substrate with a non-round cross-section having a channel;

FIG. 9B is a cross-sectional view of a non-round substrate having a channel and fibers;

FIG. 10A illustrates a view of the ends of a cleaning device in a loop configuration, in accordance with an exemplary embodiment;

FIG. 10B illustrates a schematic of a linking device holding the ends of a cleaning device together;

FIG. 10C illustrates a view of another exemplary embodiment of the ends of a cleaning device in a loop configuration;

FIG. 10D illustrates a schematic of a linking device of the exemplary embodiment illustrated in FIG. 10C holding the ends of a cleaning device together;

FIG. 11A illustrates a view of an exemplary embodiment of a cleaning device; and

FIG. 11B illustrates an end-view of the exemplary embodiment illustrated in FIG. 10A.

FIG. 12 illustrates a cleaning device in a partially straightened and partially coiled configuration in accordance with an exemplary embodiment.

FIG. 13 illustrates a cleaning device in accordance with an exemplary embodiment.

FIG. 14 illustrates a schematic of an endoscope pre-cleaning device having plugs.

FIG. 15 shows a schematic of a portion of a cleaning device having a woven cover in accordance with an exemplary embodiment.

FIG. 16 shows a process for weaving a tube over a substrate in accordance with an exemplary embodiment.

FIGS. 17A and 17B illustrate a schematic of an expandable endoscope cleaning device in accordance with an exemplary embodiment.

FIGS. 18A and 18B illustrate a schematic of an expandable endoscope cleaning device in accordance with an exemplary embodiment.

### DETAILED DESCRIPTION OF EMBODIMENTS

The following description is intended to convey a thorough understanding of the embodiments by providing various embodiments and details involving a cleaning device that utilizes a standing external spine over at least a portion of its length. This configuration could also employ antimicrobials to enhance its cleaning/pre-cleaning function. This device could also incorporate a loop and cinch assembly to provide a conveyor belt approach to cleaning, and this approach could also be effective with brush designs where the abrasive elements and/or abrasive features are primarily employed at one or both ends. It is understood, however, that the invention is not limited to these specific embodiments and details, which are exemplary only. It is further understood that one possessing ordinary skill in the art, in light of known devices, systems and methods, would appreciate the use of the invention for its intended purposes and benefits in any number of alternative embodiments.

The general inventive concepts will now be described with occasional reference to the exemplary embodiments of the invention. This general inventive concept may, however, be embodied in different forms and should not be construed as limited to the embodiments set forth herein. Rather, these embodiments are provided so that this disclosure will be thorough and complete, and will fully convey the scope of the general inventive concepts to those skilled in the art.

Unless otherwise defined, all technical and scientific terms used herein have the same meaning as commonly understood by one of ordinary skill in the art encompassing the general inventive concepts. The terminology set forth in this detailed description is for describing particular embodiments only and is not intended to be limiting of the general inventive concepts. As used in this detailed description and the appended claims, the singular forms “a,” “an,” and “the” are intended to include the plural forms as well, unless the context clearly indicates otherwise.

Unless otherwise indicated, all numbers, such as for example, numbers expressing measurements or physical characteristics, used in the specification and claims are to be understood as being modified in all instances by the term “about.” Accordingly, unless otherwise indicated, the numerical properties set forth in the specification and claims are approximations that may vary depending on the suitable properties sought to be obtained in embodiments of the invention. Notwithstanding that the numerical ranges and parameters setting forth the broad scope of the general inventive concepts are approximations, the numerical values set forth in the specific examples are reported as precisely as possible. Any numerical values, however, inherently contain certain errors necessarily resulting from error found in their respective measurements.

Disclosed herein is a cleaning device for an endoscopic device. An exemplary embodiment of the cleaning device disclosed herein has several features that improve upon the effectiveness of known pre-cleaning practices. First, by design, its geometry and material can generate significant surface contact with the interior surface of the endoscope channel that can loosen stubborn debris present in the channel. The interior surface of the endoscope channel can have its entire length in contact with the abrasive elements of the exemplary embodiment, due to the length and flexibility of the bristles. This equates to 100% of the length of the endoscope channel in contact with bristles at one time, whereas other devices may provide contact over six (6) inches or less at one time (often, less than one inch), in an endoscope channel that is for example, 90 inches long.

Next, antimicrobial additives located in the catheter (substrate) or bristles or both work with any residual moisture to stunt biological growth and can eliminate microbes remaining in the channel after the endoscope has been used in a procedure. By leaving the device in the channel until the endoscope is transported to a central cleaning room, the antimicrobial action can continue to work until the final cleaning occurs. The mere action of inserting this abrasive member into the channel will help loosen any residual debris. As it continues to reside in the channel, it interferes with the ability of the debris to reattach itself to the endoscope channel surface. In fact, since the cleaning device has higher surface energy than the endoscope channel, the debris may be preferentially drawn to it. The physical removal of the device can further initiate abrasive action throughout the channel, removing a significant amount of debris and contaminants that are caught and are dragged out by the cleaning device, rather than remaining in the endoscope channel. Finally, having this cleaning device reside in the channel prior to and during its transportation to the central cleaning room is one extra safeguard to indicate that the endoscope is dirty and needs to go through the high-level disinfection process that is commonly performed using an automatic endoscope reprocessor (AER). The design features of the device can also help deliver a disinfectant or other cleaning product to the interior of the channel.

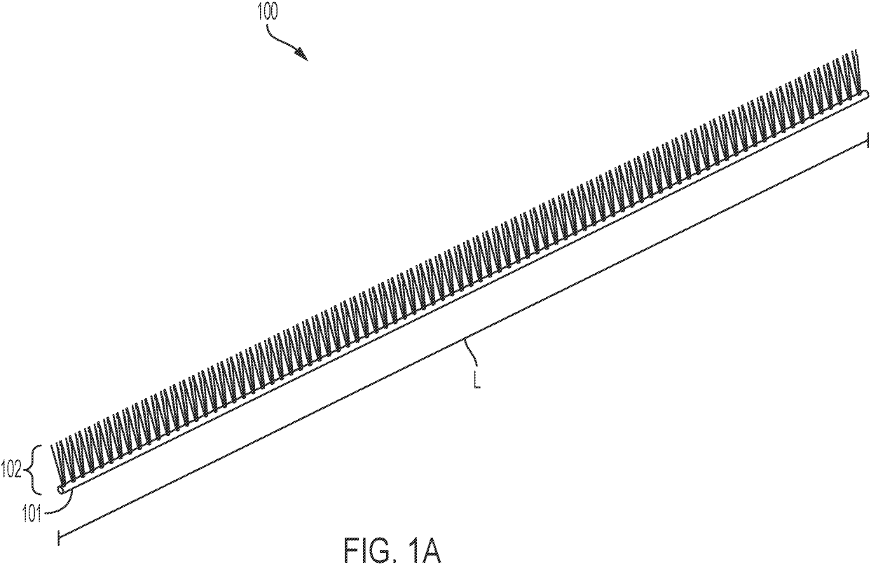

FIG. 1A illustrates a perspective view of a cleaning device 100 in a straightened configuration in accordance with an exemplary embodiment. The cleaning device 100 can have a substrate 101 that extends along the length L of the device 100 and abrasive elements 102 that can be attached to the substrate and can also extend along the length of the device 100. The device can have a total length at least the same length or longer than an endoscope channel length. A typical endoscope channel device has a length of about 90-92 inches, so the device in the exemplary embodiments described herein can have a length of at least 90 inches, for example 90-95 inches. The substrate can be an extruded shaft or catheter, to which is added an external spine element. The external spine can be an abrasive element. The abrasive elements can be standing bristles, yarn, fabric, fins, or other material, and can have properties as described in accordance with the exemplary embodiments described herein. In an exemplary embodiment, the abrasive elements can be Nylon or other polymeric bristles, and can be made of Nylon-6,6. The device 100 can provide an abrasive cleaning action to a channel of an endoscope, such as an accessory channel.

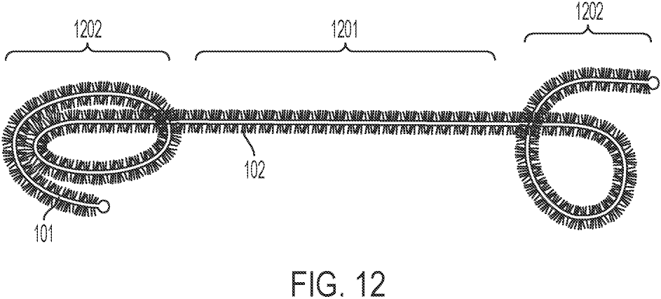

FIG. 12 illustrates an exemplary embodiment of the endoscope cleaning device 100 having a straight component 1201 and coiled end components 1202. As with the embodiment described in FIG. 1, the embodiment in FIG. 12 has a substrate 101 that extends along the length of the device 100 and abrasive elements 102 that can be attached to the substrate and can also extend along the length of the device 100. The substrate 101 can be a center core made of a wound wire, fiberglass, polymer, or other materials. Abrasive elements 102 can extend outward from the substrate. The substrate and the abrasive elements can extend along the entire length of the device. In another embodiment, the abrasive elements can extend along portions of the substrate, grouped in bunches as described herein.

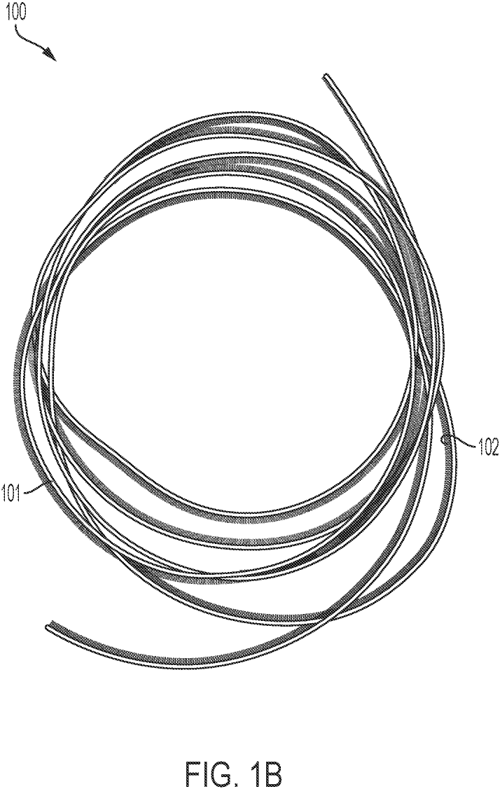

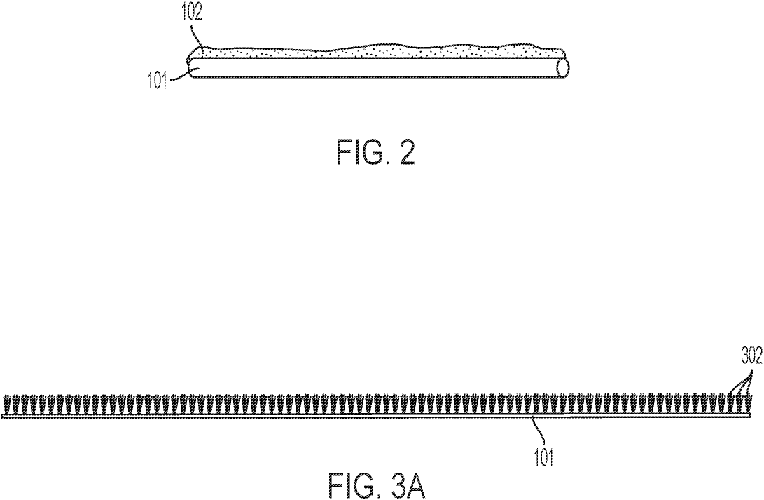

FIG. 2 illustrates a schematic of an exemplary embodiment of an endoscopic cleaning device, where the substrate extends along the length L of the device, and where the abrasive element 102 is attached to the substrate and can be positioned anywhere along the length L of the device. In FIG. 2, the abrasive element 102 is a fin. The abrasive element in FIG. 2 is not limited to a fin, but can be one of any of the exemplary embodiments described herein, and the substrate can take any one of the forms described herein. The attachment of the substrate to the abrasive element can be by any of the means described herein. The backside of the substrate can also be altered, through mechanical, heat, or other means, to have an abrasive feature that can also assist in the removal of debris which may otherwise remain stubbornly attached to the endoscope channel. The abrasive feature can be a physical alteration of a surface of the substrate. As shown in FIG. 3B, an exemplary embodiment is a substrate with an abrasive feature, wherein the substrate can have grooves and edges that have been mechanically created at any location on the outer surface of the substrate. In an embodiment having a circular cross-sectional area, the grooves and edges can be mechanically created anywhere circumferentially on the substrate. In an exemplary embodiment, abrasive features can be added 180-degrees opposite the standing bristles and/or fibers. In other embodiments, the abrasive feature can be, for example, at an angle of 45, 90, or 135 degrees from the standing bristles and/or fibers.

The substrate 101 can have properties that induce it to twist and rotate as it is moved through an endoscope channel. In its resting, unused configuration, the length L of the device can be in a coiled configuration, as illustrated in FIG. 1B. This twisting and/or rotating assists the abrasive elements in making more continuous and firmer contact with the interior surface of the endoscope channel during its insertion and extraction. When in motion, the abrasive elements and abrasive feature, if present, contact numerous portions of the interior surface of an endoscope channel in a cork-screwing manner, resulting in effective cleaning. The material properties of the substrate add to this ability to increase the amount of contact between the abrasive element and the interior surface of an endoscope channel. This increases the effectiveness of the cleaning performed in situations where an endoscope may not have been cleaned immediately post-use, even though pre-cleaning immediately after use is the recommended standard. It also improves the efficiency on more stubborn debris that is not fully cleaned away by flushing fluid alone, even when this flushing occurs immediately post-procedure. This cleaning device, with its abrasive, yet non-damaging features, can help ensure effective cleaning.

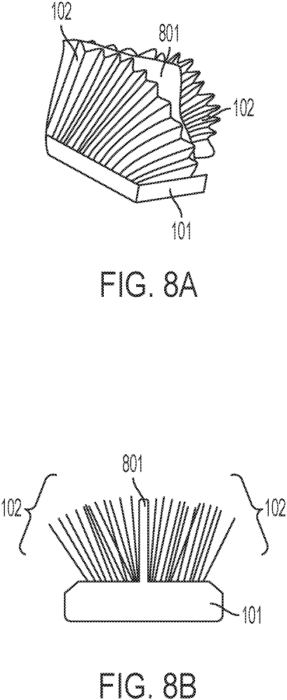

An abrasive element can be made of yarn, tufted yarn, and/or other similar fabric material, nylon bristles, a sponge-like material having absorptive and/or compressive properties, and/or an extruded fin or fins of polymeric material, and/or combinations thereof. The abrasive elements can be stiff enough to abrade the debris from the interior surface of a channel, yet flexible enough to ensure adequate surface coverage as well as not to generate inappropriately large frictional forces as the device is moved through the endoscope channel. The substrate and abrasive elements can each have a Durometer range of or about 30-95 Shore A. In addition, there are several different fiber materials to choose from, for example, Nylon, Polyester, and Polypropylene, to name a few. The behavior of these fibers and their interaction with the endoscope channel wall, also depends upon the diameter of each fiber, which can range from 0.003 inch to 0.040 inch. The height of an abrasive element can range from 0.025 inch to 0.475 inch and the number of fibers per inch can also influence flexibility and interaction with the endoscope channel. Fiber height can range from 0.025 inch to 0.475 inch. The height of individual abrasive elements, such as fibers, can be varied, where some fibers have a greater height than others. Fibers density in the form of fibers per inch of length can also be varied and can range from 20 to 800 fibers per inch. For example, a 0.005 inch Nylon monofilament can have a density ranging from 60 to 600 fibers per inch. Testing has indicated that for abrasive cleaning and induced rotation within the endoscope channel, varying the height of the bristles results in a change in stiffness of the fibers. The shorter fibers are more stiff and rigid, and clean more aggressively, than the longer fibers, whereas the longer fibers lay down more easily, increasing the surface area of the interior surface of the endoscope channel with which they interact. Having a variation in stiffness in the fibers helps induce more device rotation during insertion and removal so that the entire channel surface is contacted with some of the shorter, stiffer bristles. This can also be accomplished during the inline production, where the cutting equipment position is changed creating a variety of bristle heights along the device length. Longitudinal gaps 303 between the bristles can also provide a reservoir for debris to collect for removal. The bristle characteristics, such as material, and cross-sectional shape and diameter, also influence the overall performance. Bristles can be polygonal or round in cross-section, as can the substrate. There are also a variety of endoscope channel diameters that influence which dimensions and features should be used. The abrasion element can be felt- like (for example by using a tufted yarn) and have a width W that extends along at least a portion of the substrate (see FIG. 7A). The geometry of the device and its abrasive elements are such that fluids and debris are readily contained therein and get removed with the device upon its extraction from the endoscope channel.

FIG. 3A is a side view of the device 100 illustrated in FIG. 1. The abrasive element 102 can be a small grouping of bristles 301 attached to the substrate 101 at one end of a small grouping of bristles 301 and extending away from the substrate so that the opposite end of each bristle is unattached to anything. As illustrated in FIG. 3B, more than one small grouping of bristles 301 can be grouped in small bunches 302 inserted together in the substrate 101. FIG. 3B also shows one form of abrasive feature 103 where grooves and edges have been added, in this case, to the edge opposite the bristles. FIG. 3C is a close-up of the encircled area of FIG. 3B, illustrating the detail of the bristles in accordance with one embodiment. The bristles themselves can be cylindrical in shape with flat ends, but are not limited to this shape. In an exemplary embodiment, the bristles are all one length, but the bristles can be of two or more lengths.

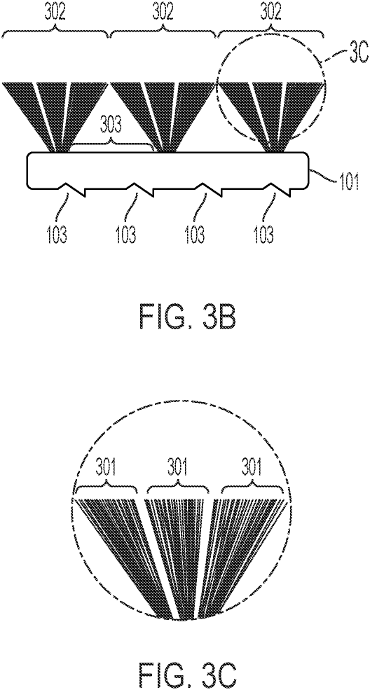

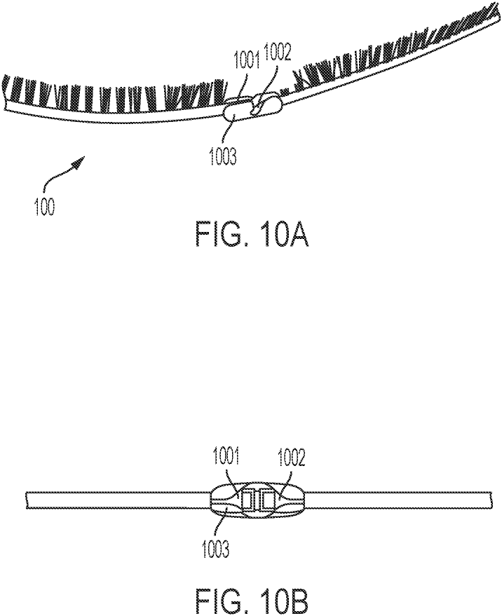

In the exemplary embodiment of FIG. 1, the substrate 101 can be round and have a circular cross section, such as that illustrated in FIG. 4A, which is a cross-section taken from a section of the device 100 in FIGS. 1A and 3A. The substrate can also have a cross-section of another shape, such as polygonal or hexagonal, as described herein. FIG. 4B also illustrates that the circular cross-section of the substrate can have a trough 401, which can extend along the entire length of the substrate. The trough can have three flat surfaces, to permit insertion of the abrasive element into the substrate. FIG. 4B illustrates the cross-section of the substrate, without an abrasive element attached, to indicate the location and shape of trough 401 in an exemplary embodiment. The trough can have other shapes, such as illustrated in FIGS. 9A and 9B. In effect, this creates a trough for the abrasive element along the length of the substrate. The substrate 101 can have a diameter ranging from at or about 0.030″to 0.130″and can be chosen based upon the endoscope channel diameter and specific substrate characteristics, to allow easy and quick insertion and removal of the device 100.

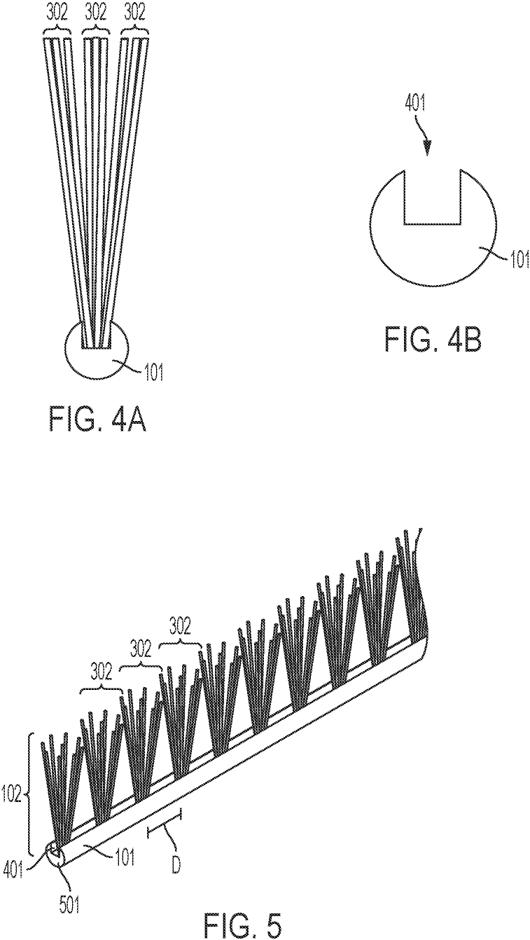

FIG. 5 is a perspective close-up view of a section of the exemplary embodiment illustrated in FIG. 1. In FIG. 5, the abrasive element can be bristles 302 clumped together, where each clump 302 can be separated from another clump 302 by a gap, indicated by distance D along the length of the substrate. Each end 501 of the substrate can be flat or can include a grasping component or connection geometry (not shown) on the end to assist the user in pushing or pulling the device 100 through an endoscope channel. Thus, the abrasive element can be a continuous line of bristles or it can be small groups of repeating abrasive spines and/or gaps in the spine.

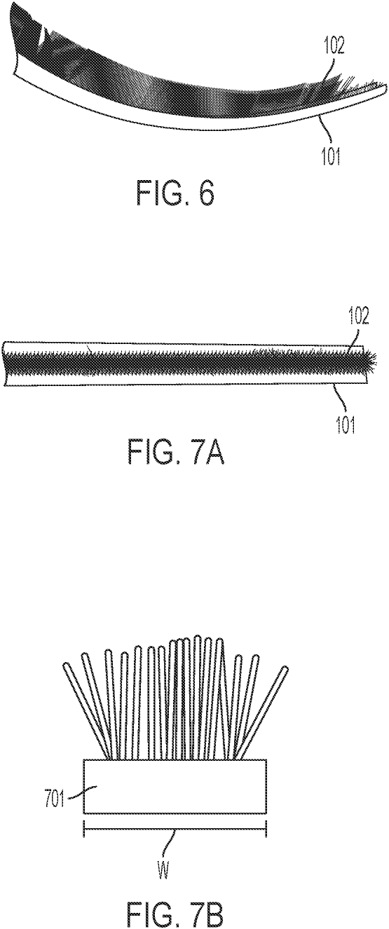

FIG. 6 illustrates a portion of an embodiment of an endoscopic cleaning device where the abrasive element 102 can be bristles in a continuous row along the length of the substrate 101. The bristles can be bonded to the substrate.

FIG. 7A illustrates a portion of another embodiment where the bristles can be continuous along the length of the substrate 101. In FIG. 7A, a top perspective view, the substrate 101 can be elongate having a length L and having a flattened cross-section shape 701, indicated in FIG. 7B. The bristles can be bonded directly to the top surface of the substrate.

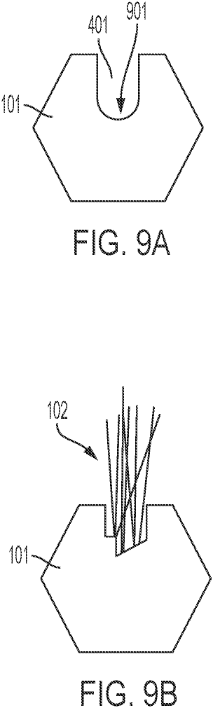

FIG. 8A illustrates another embodiment of an endoscopic cleaning device having an elongate substrate 101, and two abrasive element mediums. The first abrasive element 102 can be bristles, and a row of bristles can run along the length of the substrate on each side of the top surface of the substrate. There can be a second substrate which can be a fin 801 that is vertically positioned in the center of the substrate and runs along the length of the substrate. FIG. 8B illustrates a cross-section of the embodiment having two abrasive element mediums. In this instance, in addition to or instead of the fibers, a thin film that is polymeric and/or fabric in nature can be bonded to the substrate, providing additional surface contact and stability while residing in the endoscope channel.

The device can be symmetrical along its length. The device can also have end portions with increased or decreased stiffness compared to that of the majority of the length of the device. This can be advantageous if there are areas of the endoscope that respond more favorably to a different stiffness or geometry. For example, there may be a region that is difficult to access that can be more effectively reached by an area that is significantly more flexible. An end of the device with decreased stiffness can make the end portion more compliant for enhanced responsiveness to curvature of the endoscope. The remainder of the device can be less flexible to prevent difficulty of insertion over the distance it must travel. Conversely, the device may respond better if the proximal end is more rigid and does not kink over more aggressive brushing. The stiffer proximal end can function as a handle. In the exemplary embodiments the distal and/or proximal portions of the device can be modified to alter their physical properties to differ from the bulk length of the device. These features can be incorporated to either one or both ends of the device.

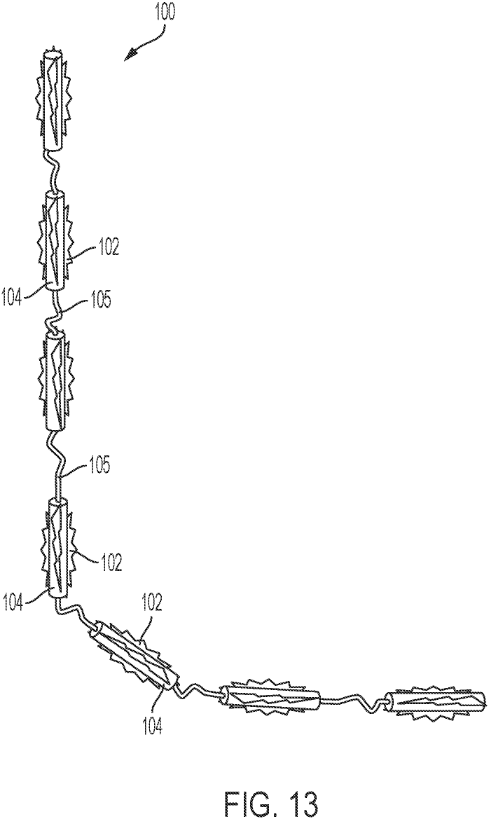

FIG. 13 illustrates an exemplary embodiment of an endoscope cleaning device having certain portions that are more flexible and certain other portions that are more rigid. The device illustrated in FIG. 13 can have semi-rigid segments 104, from which abrasive elements 102 extend. The semi-rigid segments can be made of polypropylene or other materials. The semi-rigid segments can be linked together by flexible linking segments 105 which can be made from a combination of elastomeric, fabric, soft spring, or other materials. The flexible linking segment 105 can be a tube that possess absorbency and/or a compressible surface that can have a rigid core. In the alternative, the flexible linking segment can have a bore running longitudinally though it, or can be tubular, and can be placed over a rigid core. When placed in an endoscope channel, the abrasive elements of the flexible linking segment creates enough intimate contact with the endoscope channel so as to loosen or pick up any bacteria still in the endoscope channel. As explained in further detail below, the cleaning device can have antimicrobial properties in its materials. The antimicrobials can kill bacteria on contact and also prevent the reformation of microbe and generate some abrasive action during insertion and removal of the device. With the antimicrobial treatment in contact with the interior surfaces of the channel, the surfaces can be disinfected and remain so during the entire time the device is inside the channel prior to the endoscope undergoing a high-level disinfection or sterilization process. The device, by making physical contact with the debris, can also carry debris out of the channel with it upon removal. The anti-microbial properties can be in the abrasive elements 102 and/or in the semi-rigid segments 104. The combination of the semi-rigid segments and the flexible linking segments allows the user to “inch-worm” the device through the endoscope channel to create abrasive action with the interior of the endoscope channel. The flexible linking segments of the device can be springs. As with springs, the flexible linking segments can be longitudinally compressed and stretched. When a flexible linking segment is compressed, energy is stored within the segment. The stored energy is then translated to kinetic energy, evidenced by the longitudinal expansion of the flexible linking segment. The flexible linking segments can be stretched and compressed alternatively; that is, when a flexible linking segment on one side of a semi-rigid segment is stretched and/or elongated, the flexible linking segment on the other side of that semi-rigid segment is compressed. With the “inch-worm” technique, a second semi-rigid segment can be moved closer to a first semi-rigid segment, in a direction distal from the user, prior to moving the first semi-rigid segment in a further distal direction from the user. This moves the semi-rigid segments within the endoscope channel, which scrubs debris from the interior endoscope channel wall.

In an exemplary embodiment, the abrasive element can be short bristles and they can be stiffer. The bristles in this embodiment can be short enough, for example, 0.025 inches so that they are stiff enough to not bend when the device is inserted through the endoscope channel. The fibers can also be excessively long (greater than the channel diameter), where they have little rigidity and instead flex and curl in the channel, providing less abrasive action but more surface contact with the channel wall.

An exemplary embodiment of a method for using the device is for pre-cleaning an endoscope immediately after use, while still in the procedure room, and can include the following steps. First, flushing a large volume of water through the endoscope, followed by flushing air through the endoscope to remove the bulk of the residual water. After these steps, biological material and debris still typically remains in an endoscope channel. The pre-cleaning device, which can have antimicrobial properties in its materials, can then be utilized. By using a pre-cleaning device with antimicrobial properties immediately after flushing, a user is able to skip the step of mixing and flushing with detergent or enzymatic cleaner, and also provide a channel that is easier to clean than traditional pre-cleaning techniques. The antimicrobials can kill bacteria on contact and also prevent the reformation of microbe and generate some abrasive action during insertion and removal of the device. With the antimicrobial treatment in contact with the interior surfaces of the channel, the surfaces can be disinfected and remain so during the entire time the device is inside the channel prior to the endoscope undergoing a high-level disinfection or sterilization process. The device, by making physical contact with the debris, can also carry debris out of the channel with it upon removal.

It should be noted that there are various methods to add antimicrobials, for example, resin additives added prior to extrusion and/or injection molding, as well as dip or spray coating, etc. Antimicrobial and/or bacteria growth inhibiting action can also be achieved by incorporating nano-sized texture and surface geometry features on the device. For the preferred embodiment, the device is inserted into the endoscope and left in the endoscope channel, until the endoscope is ready to receive its final cleaning. The device can depend on friction to maintain its proper position during any storage and/or transport of the device. Any moist debris or water in the channel will activate the antimicrobials making subsequent cleaning more efficient.

The device can then be pulled or pushed out the endoscope channel. It can be rinsed, used for some additional manual cleaning, or discarded. The endoscope will then undergo the standard cleaning per the SGNA Guideline, followed by either AER equipment cleaning or sterilization. The device can also be utilized for standard manual cleaning prior to AER while the endoscope is submerged in the detergent and/or disinfectant solution, or any known commercially available product used for cleaning endoscopes and other surgical devices. Once removed from the channel, the device can be rinsed, in the detergent solution to remove any visible debris before retracting and reinserting it. The device can be pushed or pulled through the channel again any number of times, such as three for example, until there is no debris visible on the brush.

In another exemplary embodiment, the device can be linked together in a conveyor belt fashion, to aid in its ability to be pulled back and forth to remove debris before the device is removed.

Abrasive features can also be added in the form of grooves cut into the substrate, either in addition to, or in place of, any of the other abrasive elements described herein. It should be noted that these grooves can be produced in a manner where they have directional asymmetry, meaning that the aggressiveness of the edge effects is different depending upon which direction it is inserted. The delta of this differential can be tailored by the geometry and could be produced in a manner where there is no directional difference. If it is desired to have a directional effect, an insertion handle or an additional item of utility (such as a valve brush, like US Endoscopy’s Double-Header Brush), could be added to the appropriate end. It is also advantageous to have a substrate with a cross-sectional geometry other than round, to provide stability during manufacture as well as provide additional abrasive edges. FIGS. 9A and 9B illustrate a hexagonal cross-sectional substrate geometry. In FIG. 9A, the cross-sectional area of the substrate 101 is hexagonal, and has a trough 401 that can run along the length of the substrate 101. The trough 401 can also have a shorter length, so that it is a notch, and there can be a plurality of notches along the length of the substrate. The trough can have a curved bottom surface 901, as illustrated in FIG. 9A, and can have any other surface shape that permits an abrasive element to be positioned within the trough. FIG. 9B illustrates the substrate 101 with a hexagonal cross-sectional shape, with abrasive element 102 attached. The abrasive element can be bonded to the substrate with adhesive or any process of attachment described herein. Abrasive cuts can extend the full length or can extend a majority of the length of substrate, without having abrasive cuts on one or both ends, in a handle region on the ends of the substrate.

The device 100 can be manufactured using an in-line manufacturing process. The substrate can be extruded. A cut or other opening can be made in the substrate into which the abrasive elements can be inserted. Other means for attaching the abrasive elements can also be used, for example, with an adhesive.

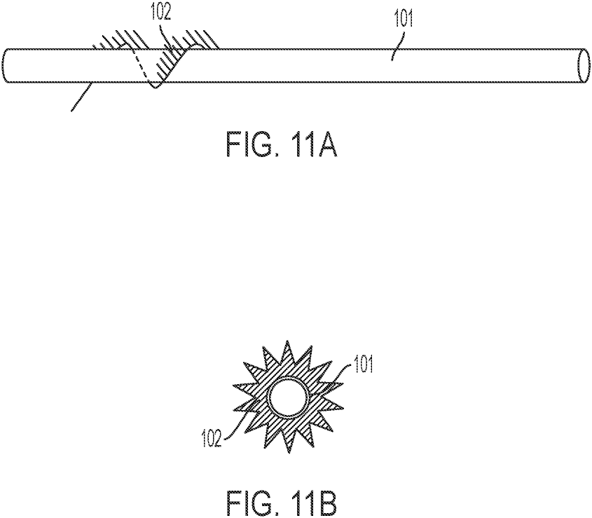

An exemplary embodiment of a cleaning device could also have an abrasive element wrapped around a substrate. FIG. 11A illustrates an exemplary embodiment of a substrate 101 having an abrasive element 102 wrapped around it, similar to a stripe wrapped around a barber pole. The abrasive element can be one or more abrasive elements including woven fibers and/or microfibers, and/or bristles. The substrate can be a hollow catheter. The abrasive element 102 can project outwardly from the substrate, in a direction perpendicular to a longitudinal axis of the substrate and can rotate around the entire outer surface of the substrate 101. The abrasive element 102 can be bristles of the same height or of varying heights. FIG. 11B illustrates an end-view of one of the possible exemplary embodiments of FIG. 11A. Depending upon the height, spacing, and other characteristics of the abrasive element 102, the end-view can be as illustrated in FIG. 11B or it can have more or less uniformity around the outer surface of the substrate 101. In an exemplary embodiment, bristles having a uniform height can be wrapped around the substrate, and the end-view can have more uniformity than that depicted in FIG. 11B. In a wrapped- around embodiment such as that illustrated in FIG. 11B, the lesser amount of surface contact of the abrasive element on the interior channel surface reduces friction while still maintaining sufficient contact between the abrasive element and the interior channel surface for antimicrobial activation, and acts like an auger to provide more extensive and aggressive debris removal.

In another exemplary embodiment, the substrate abrasive element can be added in-line or in a second stand-alone automated process. These abrasive elements could be created when the substrate is manufactured (for example by extrusion or pultrusion), or added through secondary means such as ultrasonic, heat, crimping or other mechanical means. One example of this would be to feed the catheter into a piece of equipment that can cut grooves and/or create standing ribs, producing an abrasive outer surface to enhance abrasive cleaning and/or increased surface area contact.

In addition, because there are features that induce rotation as the device is advanced through the endoscope channel, there is no need to be concerned with a particular orientation of the bristles with respect to the channel during initial device insertion. The relatively long and flexible fibers can freely contort, ensuring adequate contact with the channel wall.

Another exemplary embodiment of a method of using the device is as a cleaning brush, which can also be performed at any time, including immediately after use of an endoscope. The device 100 can be used for this utility by inserting it into an endoscope channel at a proximal end and continually feeding it into the channel until it exits the distal end of the endoscope. The movement of the device through the endoscope channel causes the abrasive element of the device to contact the interior surface of the endoscope channel and remove debris from the interior surface. For adequate cleaning, this can be repeated as many times as necessary, for example three times. In another exemplary embodiment, the device can be pulled through an endoscope channel from the distal end of the channel by pulling on the distal end of the device 100. Each exemplary embodiment described herein can be used either by being pushed or pulled through an endoscope channel.

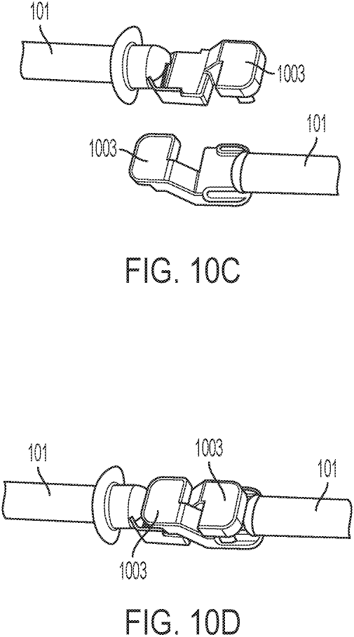

In another exemplary embodiment, the ends 1001, 1002 of the device can be configured so that they are capable of being linked together as illustrated in FIGS. 10A and 10B, to form a loop, and then subsequently pulled apart. The ends 1001, 1002 can be linked together with a linking element 1003. In this embodiment, the device’s total length would need to be a little longer than twice the length of a typical endoscopic channel device. A typical endoscope device length is, for example, 90-92 inches, so the device of the linked embodiment could be at or about 196 inches, to be linked and used effectively. The linking element can be similar to, and used in a manner similar to, a ball chain connector element used to connect two ends of a ball chain, but the linking element can also be any piece capable of being removed from at least one end of the cleaning device after it has been attached to both ends of the cleaning device. The linking element 1003 can be crimped onto one end 1001 of the cleaning device. The other end 1002 of the linking device is capable of being inserted into the linking device 1003. This allows the device to form a reel and to be operated in a continuous manner (like a conveyor belt), which can allow the user to quickly and seamlessly reverse directions for enhanced cleaning effectiveness. In another exemplary embodiment, illustrated in FIGS. 10C and 10D, the linking element 1003 can be a latch connector, and there can be a latch connector on each end of the device. Each end of the device can have an identical latch connector. The latch connectors connect to each other to form a reel. The latch connector can have a hook shape, and the hooks link together, forming a latch connection. FIG. 10C depicts the latch connector type of linking element 1003 in a disconnected position, and FIG. 10D depicts a latch connector type of linking element in a connected position. A distal portion of each latch connector overlaps a proximal portion of the other latch connector.

The linking element feature can also help keep the endoscope in a coiled orientation, should this be desired. The device can be twisted prior to connecting the ends, to create a twist that will remain during use of the device. When the ends 1001, 1002 of the device are linked together, the device can be continuously moved in either direction, or in alternating directions, through the channel to clean it. It should be noted that the device can still be used in the more traditional manner, should the user not choose to link the ends together. The linking of ends together can allow the efficient operation of an automatic insertion. For example, a device with counter-rotating wheels can be used to push and/or pull the linked assembly through the endoscope channel, providing a method for the user to insert the device other than manually pushing and/or pulling the device.

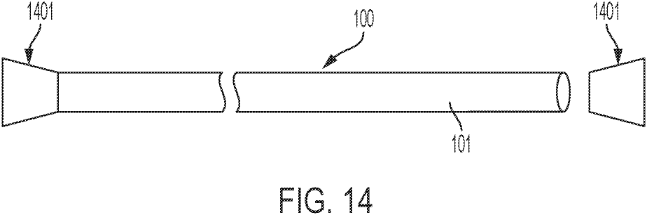

FIG. 14 illustrates a schematic of another exemplary embodiment of an endoscope cleaning device 100. In this embodiment, the substrate 101 can be an extruded shaft or hollow catheter. However, the substrate is not limited to an extruded shaft or hollow catheter, and can be any substrate described herein. The device can have plugs 1401 at each end of the substrate 101 to help anchor the device 100 within the endoscope channel. One or both of the plugs 1401 can be removeable. FIG. 14 illustrates one plug that is attached, and one plug that has been removed, from the substrate. When the device is placed in an endoscope channel, the plugs maintain contact with the opening at each end of the endoscope channel around the entire circumference of the endoscope channel openings. The plugs 1401 can retain moisture within the endoscope channel that encourages anti-microbial action and removal of bioburden within the endoscope channel.

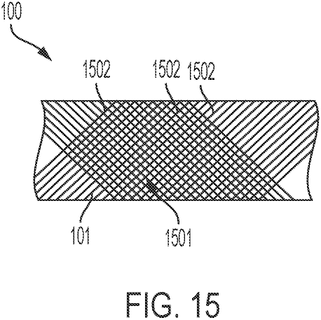

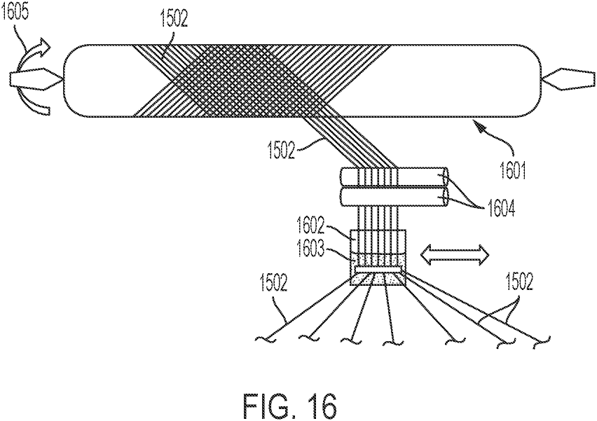

FIG. 15 illustrates a schematic of a portion of an endoscope cleaning device having a rigid core substrate 101 and a woven tube 1501 made of woven fibers 1502 surrounding the substrate. The woven tube can be a woven fabric tube having absorbent properties, and/or a compressible, irregular surface. The tube is placed over a rigid core substrate. The tube can be formed by weaving individual fibers 1502 onto a mandrel 1601. As illustrated in FIG. 16, the fibers that are woven together can pass through a vessel 1602 with resin 1603, then the resin-coated fibers can pass through a pair of nip rollers 1604, to create uniformity in the resin coating. Then the fibers can be woven onto a mandrel 1601 into a tubular shape. The mandrel rotates as indicated by arrow 1605. The woven tube can then be positioned over a substrate. In another exemplary embodiment, the substrate can be covered with a heat-shrink element that gives the device the desired external surface that will interface and create contact with the endoscope channel. The external surface created by the woven fibers and/or heat shrink element can be used to maintain contact with interior surface of the endoscope channel and can be used to dislodge and remove debris from the endoscope channel. The woven tube and/or heat-shrink element can also have anti-microbial additives. In other exemplary embodiments, the substrate can be any tubular substrate described herein.

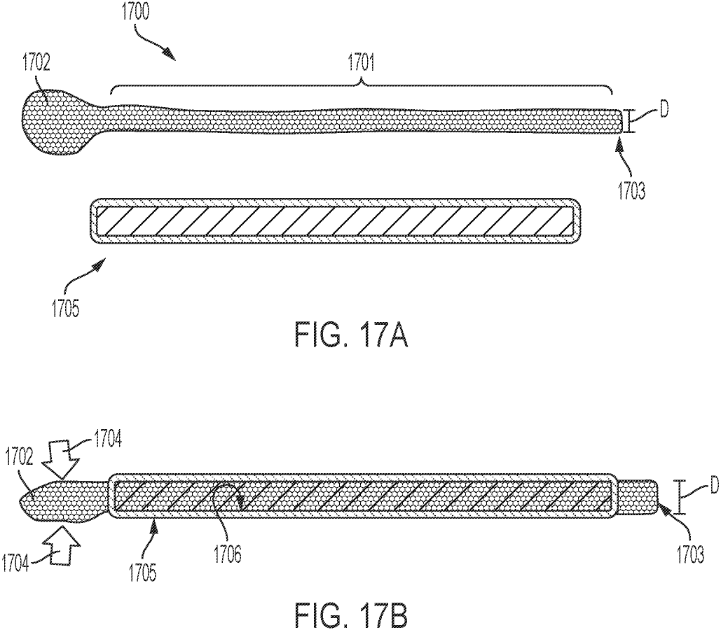

Additionally, the tubular substrate can be created in a hollow manner where the substrate is sealed on at least one end. FIGS. 17A and 17B illustrate an exemplary embodiment of an endoscope cleaning device 1700 that is an elongate tubular substrate 1701 having a sealed bulbous end 1702 and an open end 1703 for inflation. The open end 1703 can have a fitting (not shown) for inflation and/or can have a closure to prevent air from escaping once the substrate has been inflated. The bulbous end 1702 can have a bulbous shape, or other volume, that is larger in cross-sectional area than the elongate tubular substrate 1701 of the endoscope cleaning device, as illustrated in FIG. 17A. When inflated, the bulbous end expands to an inflated volume (shown in FIG. 17A), which is greater than its uninflated volume. The endoscope cleaning device can be placed into the endoscope channel 1705, as illustrated in FIG. 17B. The elongate tubular substrate 1701 has a length sufficient for extending along the length of the endoscope channel such that the bulbous end extends beyond an end of the endoscope channel. Upon being inflated, the open end 1703 of the endoscope device can be sealed, to retain the air within its interior. The expanded bulbous end can be compressed, with the compression indicated by arrows 1704. Compression of the bulbous end 1702 causes the air within the endoscope cleaning device to be pushed into the elongate tubular substrate, causing the diameter D of the elongate tubular substrate to expand. The expansion of the elongate tubular substrate portion of the bladder causes the exterior of the elongate tubular substrate to make intimate contact with the interior surface 1706 of the endoscope channel. This contact with the wall can be used to remove debris and disinfect the endoscope channel, similar to the other embodiments described herein.

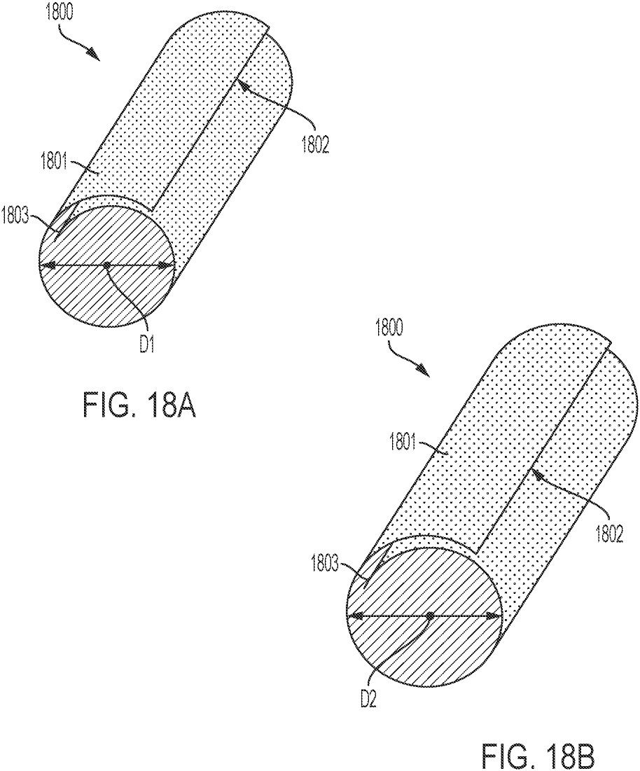

FIGS. 18A and 18B illustrate a schematic of an exemplary embodiment of an endoscope cleaning device 1800 that is an elongate hollow substrate 1801 that is formed by rolling and/or twisting a substantially flat piece of material into a tubular shape. FIGS. 18A and 18B illustrate a portion of the entire length; the device 1800 can be as long as or longer than an endoscope channel. The device is not required to be a flat sheet, but can have an outward facing surface 1801 that is textured, and/or can contain abrasive features, such as but not limited to the abrasive features 103 cut into the substrate of the embodiment illustrated in FIG. 3B. The substrate has two long edges, the length of each being as long as or longer than the endoscope channel. When the substrate is rolled to form the hollow tubular structure, the first long edge, which is an exterior edge 1802, is on the outside of the tubular shape, such that it overlaps with the material of the substrate and can come in to contact with the interior wall of an endoscope channel, when the device 1800 is placed inside an endoscope channel. The second long edge is an interior edge 1803, and is covered by the overlap of material of the substrate when sheet is rolled to form the elongate tubular edge. The edges 1801, 1802 are not fixedly secured to any other portion of the device 1800. This allows the device to expand once it is inserted into the endoscope channel, by having the portion of the substrate that is near the exterior edge 1802 slide over the portion of the substrate that is near the interior edge 1801. FIG. 18A illustrates the device 1800 in a constrained configuration, having a first diameter D1. FIG. 18B illustrates the device 1800 in an expanded configuration, such that is radially expanded to a second diameter D2, where D2 is greater than D1. The device is inserted into an endoscope channel in its constrained configuration, and then is expanded. The expansion of the device allows the outward facing surface 1801 to make intimate contact with the interior wall of the endoscope channel, to remove debris and/or provide antimicrobial properties, similar to the other embodiments described herein.

In another exemplary embodiment, the device can be configured so that one or both ends can be linked to stand-alone device(s) that impart vibrational and or translational energy down the axis of the device to enhance cleaning. This imparted energy can help loosen stubborn debris and result in more effective cleaning and less effort by the user. This is another instance where increased contact of the device with the interior surface of an endoscope is advantageous. Energy is transmitted more efficiently when the bristles of the device are in the scope for the entire device length as in the exemplary embodiments described herein, because vibrational and/or translational energy imparted to the device does not have to travel the entire length of the device before it can be transmitted to the bristles.

The methods of using the exemplary embodiments of the device described herein can be used together; for example, the device can be used to both remove debris from the endoscope channel and to apply a disinfectant to the channel. In an exemplary embodiment of using the device, the device can be pushed or pulled through the endoscope channel one or more times, and then left in place the last time it is placed in the channel during the pre-cleaning method, until it is ready to be cleaned with AER equipment.

The methods of using the exemplary embodiments of the device described herein can be used either for pre-cleaning and/or for cleaning an endoscope.

Accordingly, the various embodiments are not to be limited in scope by the specific embodiments described herein. Further, although some of the embodiments have been described herein in the context of a specific implementation in a particular environment for a particular purpose, those of ordinary skill in the art should recognize that its usefulness is not limited thereto and that the various embodiments can be beneficially implemented in any number of environments for any number of purposes. Accordingly, the claims set forth below should be construed in view of the full breadth and spirit of the embodiments as disclosed herein. While the foregoing description includes many details and specificities, it is to be understood that these have been included for purposes of explanation only and are not to be interpreted as limitations of the various embodiments. Many modifications to the embodiments described above can be made without departing from the spirit and scope of this description.

What is claimed is:

1. A device for cleaning an elongate channel of a medical device, comprising:
an elongate substrate having a length; and
an abrasive element co-extending along the length of the elongate substrate;
wherein the abrasive element extends outward from the elongate substrate,
wherein at least a portion of the elongate substrate is flexible, and
wherein a first end and a second end of the device are capable of being linked together to form a loop.

2. The device of claim 1 wherein the abrasive element extends outward in a single direction from the elongate substrate.

3. The device of claim 1, wherein the abrasive element comprises a plurality of bristles.

4. The device of claim 3, wherein the plurality of bristles are in a continuous line.

5. The device of claim 3, wherein the plurality of bristles are grouped in bunches of 3-15 bristles, and each bunch is separated from another bunch by a gap along the length of the elongate substrate.

6. The device of claim 1, wherein the first end and the second end of the device are capable of being removably linked together.

7. The device of claim 1, further comprising a linking element configured to link together the first end and the second end of the device.

8. The device of claim 7, wherein the linking element is removably attached to at least one of the first end and the second end of the device.

9. The device of claim 7, wherein the linking element is fixedly attached to the first end of the device and removably attached to the second end of the device.

10. The device of claim 7, wherein at least one of the first end and the second end of the device is capable of being inserted into the linking element.

11. The device of claim 7, wherein the linking element comprises latch connectors on each of the first end and the second end of the device.

12. The device of claim 1, further comprising counter-rotating wheels for pushing and/or pulling the loop formed by the device through the elongate channel.

13. The device of claim 1 wherein at least a portion of the elongate substrate comprises a twist in the axial direction.

14. The device of claim 1, wherein the abrasive element comprises antimicrobials.

15. The device of claim 1, wherein the elongate substrate comprises antimicrobials.

16. The device of claim 1, wherein the elongate substrate comprises abrasive features.

17. The device of claim 16, wherein the abrasive features comprise grooves and/or edges formed on the elongate substrate.

18. A method for cleaning an elongate channel of a medical device, comprising:
providing a device having an elongate substrate and an abrasive element co-extending along a length of the elongate substrate, the elongate substrate comprising antimicrobials;
inserting the device into the elongate channel;
linking a first end and a second end of the device to form a loop;
moving the device in one direction or alternating directions through the elongate channel, wherein the abrasive element extends outward from the elongate substrate, and wherein at least a portion of the elongate substrate is flexible.

19. The method of claim 18, wherein the device comprises counter-rotating wheels for moving the device through the elongate channel.

20. The method of claim 18, further comprising a step of twisting the elongate substrate of the device prior to inserting the device into the elongate channel.
